(19) United States
(12) Patent Application Publication
(10) Pub. No.: US 2024/0218419 A1
(43) Pub. Date: Jul. 4, 2024

(54) RAPID ENUMERATION OF MICROORGANISMS

(71) Applicants: The University of Chicago, Chicago, IL (US); Duquesne University of the Holy Spirit, Pittsburgh, PA (US)

(72) Inventors: Melikhan Tanyeri, Pittsburgh, PA (US); Jing Lin, Chicago, IL (US); Mustafa F. Abasiyanik, Chicago, IL (US); Yulder D. Angarita Marmolejo, Pittsburgh, PA (US)

(73) Assignees: The University of Chicago, Chicago, IL (US); Duquesne University of the Holy Spirit, Pittsburgh, PA (US)

(21) Appl. No.: 18/289,194

(22) PCT Filed: Aug. 6, 2021
(86) PCT No.: PCT/US2021/044964

§ 371 Date: Nov. 1, 2023

Related U.S. Application Data
(60) Provisional application No. 63/184,447, filed on May 5, 2021.

(51) Int. Cl. C12Q 1/04; G06T 7/00; G06T 7/62; G06V 10/82; G06V 20/69

(52) U.S. Cl. CPC C12Q 1/04; G06T 7/0012; G06T 7/62; G06V 10/82; G06V 20/69; G06T 2207/20081; G06T 2207/20084; G06T 2207/30128; G06T 2207/30242

(57) ABSTRACT

In aspects, the present disclosure provides methods for quantifying microorganisms. In aspects, the present disclosure provides methods for identifying microorganisms. In aspects, the present disclosure provides methods of analyzing food. In aspects, the present disclosure provides methods of treating a human subject having an infection. In aspects, the present disclosure provides methods of analyzing environmental samples.

### BACKGROUND

Rapid accurate quantification of microorganisms within a sample is important in many clinical and environmental applications. The gold standard technique for quantification is using a plate count. This technique relies on the principle that viable microorganisms replicate and give rise to visible colonies on an agar plate with growth medium. Viable or live microorganisms within a sample are determined by diluting the sample multiple times, then spreading each dilution onto separate agar plates containing growth medium. The number of colonies growing on each plate are counted and averaged. The microorganism concentration in the original sample is determined by the average colony count (typically 30-300 per plate) and the dilution factor. Consistently accurate serial dilutions are important for the reliability of this method. Plate counts typically underestimate the original concentration since more than one microorganism may have landed on the same spot on the agar during spreading. In addition, microorganisms that grow in clusters or chains also prove difficult to disperse: as a result, a single colony may represent multiple microorganisms. Consequently, the counts are reported as colony forming units per milliliter (CFU/mL) rather than microorganisms/milliliter. This decades-old technique is time consuming (it takes 24-48 hours for many microorganisms), labor-intensive, and prone to error.

Another technique for quantifying microorganisms within a sample is a counting chamber. In this technique, microorganisms within a sample are counted by: i) spreading a measured volume of the sample (typically 0.2 μL) within a calibrated chamber (e.g., a Petroff-Hauser or Levy chamber) over a known area with precise ruled lines, ii) counting microorganisms over several (typically 4-9) representative areas within the chamber using a light microscope, and iii) calculating the original microorganism concentration by relating the average count, the volume of the chamber, and the dilution factor used to prepare the sample. While this is a direct counting method, it does not necessarily yield an accurate number of live microorganisms because it is not always possible to distinguish between live microorganisms, dead microorganisms, and debris of the same size under the microscope. Furthermore, the counting chamber does not work well with dilute cultures because there may not be enough microorganisms to count.

Yet another technique is the use of optical density to quantify microorganisms. In this technique, a liquid medium becomes turbid during microbial growth as microorganisms scatter light. Concentration of microorganisms can be determined by a spectrophotometer where a light beam, typically at 600 nm, transmitted through the suspension is measured by a detector. The fraction of light passing through the sample and reaching the detector (measured on a logarithmic scale) is recorded as the absorbance or optical density of the sample. As the microorganism concentration increases, the turbidity or absorbance of the sample also increases. Measuring concentration using absorbance measurements is rapid, however, only practical at high concentrations. In addition, concentrations should be verified with direct count methods (counting chamber or plate count). For this purpose, a calibration curve is generated by plotting optical density as a function of plate count. Then, the calibration curve can be used to estimate CFU values for a given optical density. Optical density measurements are linear only within a narrow range (typically OD=0.1-0.5). Furthermore, turbidity does not differentiate between live and dead microorganisms.

Therefore, there is an unmet need for a faster and more accurate way to quantify the number microorganisms with higher throughput. The present disclosure provides methods for this unmet need.

### BRIEF SUMMARY

In aspects, the present disclosure provides a method for quantifying microorganisms, the method comprising obtaining a sample containing the microorganisms; encapsulating the microorganisms in one or more first droplets of a plurality of droplets; allowing microorganism growth within the one or more first droplets of the plurality of droplets: capturing one or more images of the plurality of droplets: performing an image analysis using artificial intelligence on the one or more images, wherein the artificial intelligence was trained: and quantifying the microorganisms.

In aspects, the present disclosure provides a method of analyzing food, the method comprising obtaining from the food a sample containing microorganisms: and quantifying microorganisms in the sample as described herein.

In aspects, the present disclosure provides a method of diagnosing an infection within a human subject, the method comprising obtaining from the human subject a sample containing microorganisms: quantifying microorganisms in the sample as described herein; and determining that the human subject has an infection when the quantified microorganisms is greater than that of a human subject without an infection.

In aspects, the present disclosure provides a method of treating a human subject having an infection, the method comprising determining that the human subject has an infection as described herein: and treating the human subject for the infection.

In aspects, the present disclosure provides a method of analyzing an environmental sample, the method comprising obtaining from the environmental sample a sample containing microorganisms: and quantifying microorganisms in the sample as described herein.

Additional aspects are as described herein.

### BRIEF DESCRIPTION OF THE DRAWINGS

FIG. 1 illustrates a schematic of bacteria quantification in liquid samples using droplet microfluidics, image processing, and machine learning, according to aspects of the present disclosure.

FIG. 2A illustrates images of droplets full of bacteria and droplets that are empty (left), and the same images segmented (right), according to aspects of the present disclosure.

FIG. 2B illustrates images of droplets full of bacteria and droplets that are empty (left), and the same images segmented (right), according to aspects of the present disclosure.

FIGS. 3A, 3B, and 3C illustrate histograms of major axis length of droplets in pixels (FIG. 3A), circularity of droplets (FIG. 3B), and perimeter of droplets in pixels (FIG. 3C), according to aspects of the present disclosure.

FIG. 4 illustrates the droplet classification of FIG. 2A, according to aspects of the present disclosure. The axes represent spatial coordinates in pixels. The intensity/contrast is used to classify empty and full droplets. Droplets with a shaded/gray square is full of bacteria, whereas droplets with a black square are empty.

FIG. 5 illustrates a confusion matrix to evaluate training of Support Vector Machines (SVMs) for droplets of FIG. 2B, according to aspects of the present disclosure.

FIG. 6 illustrates a confusion matrix to evaluate binary classification accuracy of SVMs for droplets of FIG. 2A, according to aspects of the present disclosure. Specificity, sensitivity, positive predictive value (PPV), and negative predictive value (NPV) are labeled.

FIGS. 7 and 8 illustrate images of droplets full of bacteria (E. coli in FIG. 7 and S. aureus in FIG. 8) over time, according to aspects of the present disclosure.

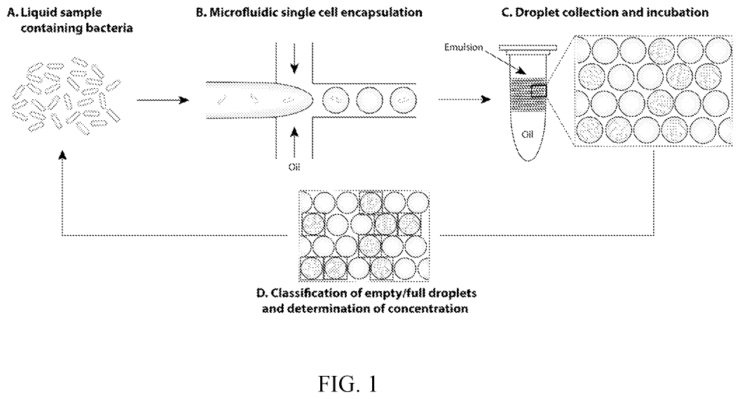

### DETAILED DESCRIPTION

In aspects, the disclosure provides a method for quantifying microorganisms, the method comprising obtaining a sample containing the microorganisms: encapsulating the microorganisms in one or more first droplets of a plurality of droplets: allowing microorganism growth within the one or more first droplets of the plurality of droplets; capturing one or more images of the plurality of droplets: performing an image analysis using artificial intelligence on the one or more images, wherein the artificial intelligence was trained: and quantifying the microorganisms.

The microorganisms may be any prokaryotic or eukaryotic single-celled organism, e.g., bacteria, protozoa, or yeast. The bacteria may be any type of bacteria, e.g., pathogenic or non-pathogenic. The pathogenic bacteria may be Mycobacterium tuberculosis, Streptococcus, Pseudomonas, Shigella, Campylobacter, Listeria and Salmonella.

In aspects, the plurality of droplets comprises an aqueous medium. In aspects, the aqueous medium contains nutrients that nurture the growth of microorganisms. Such nutrients are well-known in the art.

In aspects, the sample comprises using a microfluidic device to encapsulate the microorganisms in the one or more first droplets of the plurality of droplets. Droplet-based microfluidics is a subfield of microfluidics where an aqueous phase is segmented into individual droplets within an immiscible carrier fluid to encapsulate cells, organic molecules and reagents. Cell encapsulation provides significant benefits for microenvironmental control and sample processing. Microfluidic systems provide a platform for cell encapsulation as the droplet size is typically comparable to that of cells. The most common technique of encapsulating cells uses microfluidic channel geometries that combine co-flowing water and oil phases, where the water phase is dispersed into the oil phase and breaks into discrete water droplets. Using the common geometries such as T-junctions, flow-focusing or co-flowing intersections, the formation of droplets can be precisely regulated by adjusting the differential volumetric flow rates of the immiscible fluid phases. Methods of cell encapsulation can use either passively or actively formed droplets. Passive droplet generation takes place through the use of external pressure sources such as syringe and pressure-driven pumps. On the other hand, active droplet production occurs with the application of an active, short-duration pressure pulse.

In aspects, the microorganisms grow into a colony inside the one or more first droplets of the plurality of droplets.

In aspects, the one or more first droplets of the plurality of droplets are emulsified into an oil, e.g., an engineered oil. The engineered oil may be, e.g., mineral or fluorinated oil.

In aspects, the plurality of droplets comprises one or more second droplets of the plurality of droplets that do not contain a microorganism, and wherein the one or more second droplets of the plurality of droplets are emulsified into an oil, e.g., an engineered oil.

In aspects, the trained artificial intelligence program was trained by a method comprising analyzing one or more images, wherein each of the one or more images includes at least one of one or more droplets encapsulating microorganisms of a plurality of droplets and at least one of one or more droplets that do not contain a microorganism of a plurality of droplets, wherein the artificial intelligence is based on machine learning algorithms including neural networks and deep learning, and wherein the artificial intelligence learns whether a droplet is of the one or more droplets encapsulating microorganisms of the plurality of droplets or is of the one or more droplets that do not contain a microorganism of the plurality of droplets.

In aspects, the artificial intelligence may comprise machine learning. In aspects, machine learning may be supervised machine learning. Supervised learning begins with the objective of predicting a known output or target. It is about mapping data to known targets, given a set of examples. Supervised learning emphasizes classification, which includes selecting among subsets to best describe a new instance of data and predicting an unknown parameter. Examples of supervised learning algorithms are, e.g., pattern recognition, k-Nearest Neighbors, Linear Regression, Logistic Regression, SVMs, Decision Trees and Random Forests, and Neural Networks. The pattern recognition method can use Deep Neural Networks (DNNs). The analysis of a neural net can be compared to evaluation using a plot of true-positive against false-positive values, called Receiver Operating Characteristics (ROC), where the area under a curve (AUC) is used to demonstrate the accuracy level.

In aspects, machine learning may be unsupervised machine learning, in which patterns or groups within data are determined. Examples of unsupervised learning algorithms are, e.g., k-Means, Principal Component Analysis (PCA), Expectation Maximization, Hierarchical Cluster Analysis (HCA), and kernel PCA. Whereas supervised learning mainly addresses classification and regression problems, unsupervised learning tackles more clustering and reduced dimensionality. Patterns identified in unsupervised learning can be assessed for utility either by human interrogation or through application within a supervised learning task.

In aspects, Support Vector Machine (SVM), which is a supervised learning algorithm, may be used to divide data into two or more categories. The term “support vector” refers to the margin used by the algorithm to assess whether or not data is part of the group.

In aspects, Neural networks, also known as artificial NN, may be used. NN use multiple computation layers to simulate how a human brain interprets and draws conclusions from data. NNs are primarily mathematical models designed to manage complex and diverse information. The nomenclature of this algorithm is derived from the use of “nodes,” similar to brain synapses. A NN's learning process may either be supervised or unsupervised. The neural net is set up to learn in a supervised manner if the desired output is already targeted and implemented to the network by training data, while the unsupervised NN does not have such pre-identified target outputs and the aim is to group similar units together within a certain value range.

In aspects, a particular machine learning subfield, such as deep learning may be used. Deep learning is a perspective on data learning representations that emphasizes learning successive layers of progressively meaningful representations. The difference between deep learning and a simple NN is that the number of node layers is increased, and the total size of the network is greater, allowing for a more accurate representation of complex interrelationships.

In aspects, the image analysis using the artificial intelligence on the one or more images comprises the artificial intelligence determining that the one or more droplets encapsulating microorganisms of the plurality of droplets contain one or more microorganisms and the one or more droplets that do not contain a microorganism of the plurality of droplets do not contain a microorganism.

In aspects, a Convolutional Neural Network (CNN), which is a form of feedforward NN designed to imitate neural processes within a human brain, can be applied to image processing tasks. The architecture allows nodes to connect to a part of the input image. Convolutional blocks work within an image by moving along an image through a small window area and generating the weighted sum of the pixel values for the filter in the region and applying a non-linear transformation. Such convolutional layers can be merged with pooling (subsampling) layers, retrieving the most dominant values in the feature maps and reducing their resolution. The loop can be replicated several times until a particular resolution size of the filter map is obtained. The early phases can be programmed to resolve spatial data and convolutions can serve as special feature detectors (e.g. edges, lines), ultimately teaching the network to connect adjacent pixels in space. The result of these layers can be a low-dimensional embedded representation of an image which provides a much better representation of the image content than other methods of feature-extraction.

In aspects, the artificial intelligence determines the volume of at least one of the one or more droplets encapsulating microorganisms of the plurality of droplets and the volume of at least one of the one or more droplets that do not contain a microorganism of the plurality of droplets. In aspects, the one or more images includes the plurality of droplets, and the artificial intelligence determines a quantity of the one or more droplets encapsulating microorganisms of the plurality of droplets and a quantity of the one or more droplets that do not contain a microorganism of the plurality of droplets from the one or more images of the plurality of droplets. In aspects, the artificial intelligence learns how many microorganisms are in a droplet containing at least one microorganism, and wherein the artificial intelligence determines a quantity of microorganisms in each of the one or more droplets encapsulating microorganisms of the plurality of droplets.

In aspects, the artificial intelligence learns how many microorganisms initiated a colony, and wherein the artificial intelligence determines a quantity of microorganisms that initiated a colony by comparing one or more images of the plurality of droplets taken at predetermined time intervals.

In aspects, quantifying the microorganisms further comprises analyzing the one or more first droplets using a digital polymerase chain reaction (dPCR).

In aspects, the present disclosure provides a method of analyzing food, the method comprising obtaining from the food a sample containing microorganisms: and quantifying microorganisms in the sample as described herein. In aspects, the microorganism can be pathogenic. In aspects, the microorganism can be probiotic.

In aspects, the present disclosure provides a method of diagnosing an infection within a human subject, the method comprising obtaining from the human subject a sample containing microorganisms: quantifying microorganisms in the sample as described herein; and determining that the human subject has an infection when the quantified microorganisms is greater than that of a human subject without an infection.

In aspects, the present disclosure provides a method of treating a human subject having an infection, the method comprising determining that the human subject has an infection as described herein: and treating the human subject for the infection.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of infection in a subject. Furthermore, the treatment or prevention provided by the inventive method can include treatment or prevention of one or more conditions or symptoms of infection being treated or prevented. Also, for purposes herein, “prevention” can encompass delaying the onset of the infection, or a symptom or condition thereof.

The subject can be any mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

In aspects, the present disclosure provides a method of analyzing an environmental sample, the method comprising obtaining from the environmental sample a sample containing microorganisms: and quantifying microorganisms in the sample as described herein.

Any suitable environmental sample may be used. In aspects, the environmental sample is water. in aspects, the environmental sample is sewage.

The present disclosure provides several advantages, including, for example, the following. (a) High-precision (digitized culture): Digitized colony counting provides a more precise approach to determine the number of viable microorganisms in a sample. For example, aspects of the present disclosure eliminate the multiple dilution steps and user-dependent fluid handling and spreading on agar plates. Also, conventional plating does not necessarily circumvent landing of multiple cells on the same macroscopic spot, giving rise to a single colony. The microfluidic compartmentalization of microorganisms ensures that the microorganisms are separated randomly through the emulsification process. As a result, aspects of the present disclosure allow for development of uniform droplet colonies, thereby yielding a more accurate quantification method. (b) High-throughput (parallel sample measurements): Microfluidic platforms enable parallel analysis of multiple samples. A single microfluidic chip can be configured to include multiple droplet generation devices, thereby enabling simultaneous processing of multiple samples or generating replicates of the same sample. (c) Shorter turnaround time: As an example of microorganism growth, bacterial colonies typically go through three phases. i) Lag phase: Bacteria are metabolically active but not dividing, primarily adapting themselves to growth conditions via expression of proteins and signaling molecules required for replication. ii) Log (growth) phase: Bacteria are actively dividing by binary fission and doubling in numbers after each generation time. iii) Stationary phase: Bacterial cell growth reaches a plateau, nutrients become depleted and waste products start to accumulate. Droplet encapsulation accelerates the adaptation of bacterial cells to growth conditions due to rapid accumulation of signaling molecules within the confined droplet volume. In addition, the colony is formed within a much smaller volume, typically in the pico- to nanoliter range in contrast to macroscopic colonies formed in spread plating methods which needs extended incubation time to become visible to the naked eye. As a result, droplet colonies are expected to form sooner (within 8 hours) in comparison to conventional plating method which may require up to 48 hours of incubation. (d) Colonies can be picked: Individual droplet colonies can be transferred to culture tubes, flasks or conventional agar plates for bulk culturing and subsequent microbiological studies. While conventional methods such as plate spreading allow for colony picking, the process of colony picking and subsequent culturing is very labor intensive. Microfluidic platforms are amenable to automation and enable post colony handling, facilitating further high-throughput testing with specific colonies. (e) Post processing (antimicrobial susceptibility testing, etc.): A number of microfluidic methods have been developed to manipulate droplets. Microfluidic techniques to merge, split, sort and mix droplets enable integration of a number of sample pre- and post-processing steps. For instance, droplet merging and mixing would enable high throughput antimicrobial susceptibility testing of droplet colonies whereas droplet splitting and sorting can be utilized for colony picking to interface the microfluidic technology with conventional microbiology techniques.

In aspects, the methods of the disclosure can also be used for identification of microorganisms. In aspects, a selective/differential medium involving an enzyme, substrate or a biochemical agent (or a combination of these) can be utilized for droplet cultures, yielding a distinct signal (e.g., an optical signal such as colorimetric, fluorometric, turbidimetric, etc., or, e.g., an electrochemical signal) due to its/their interaction with the microorganism(s). A similar approach is often used with agar plates where a selective/differential agent is added to the medium. Since droplets provide a platform for growth similar to an agar plate, this approach can be used to identify or distinguish types of microorganisms. For instance, eosin methylene blue is a selective stain for gram-negative bacteria, and is selective and differential for coliforms (e.g., E. Coli). It distinguishes between microorganisms that ferment lactose (e.g., E. coli) from those that do not (e.g., Salmonella). Another example is the hemolytic reaction of red blood cells caused by certain bacteria such as Staphylococcus aureus, Pseudomonas aeruginosa, and Listeria monocytogenes. Similarly, mannitol/salt containing medium is selective for pathogenic gram-positive bacteria such as Staphylococcus aureus.

Essential reagents defining selective/differential media can be initially added to the original sample prior to droplet encapsulation and incubation. Alternatively, if these reagents are growth-inhibitive, they can be added to droplet cultures by merging two or more droplets, one containing the bacteria culture and other(s) containing the reagents which constitutes the selective/differential reagent (or media). Upon interaction between the selective/differential reagents and the microorganisms, a change in, e.g., optical (e.g., colorimetric, fluorometric, turbidimetric, etc.) or electrical (e.g., impedance, etc.), properties of the droplet culture can be induced. The change in optical or electrical properties of the droplet culture can be measured on individual or groups of droplets using relevant transduction mechanism.

As an example of identifying microorganisms, detecting the presence of Staphylococcus aureus in liquid samples can be accomplished by carrying out a droplet culture of the sample in mannitol salt broth (peptone, beef extract, sodium chloride, mannitol) and a fluorescent pH indicator (e.g., 6-carboxyfluorescein). Upon droplet culture, S. aureus would ferment mannitol and generate an acidic byproduct, thereby lowering the pH of the medium and yielding a decrease in fluorescence intensity. Following a sufficient incubation period, a combination of phase contrast and fluorescence imaging of droplet cultures would yield both enumeration and identification of the microorganism(s) based on the choice of selective/differential medium.

The following includes certain aspects of the disclosure.

1. A method for quantifying microorganisms, the method comprising:
obtaining a sample containing the microorganisms:
encapsulating the microorganisms in one or more first droplets of a plurality of droplets:
allowing microorganism growth within the one or more first droplets of the plurality of droplets:
capturing one or more images of the plurality of droplets:
performing an image analysis using artificial intelligence on the one or more images, wherein the artificial intelligence was trained; and
quantifying the microorganisms.

2. The method of aspect 1, wherein the plurality of droplets comprises an aqueous medium containing nutrients nurturing growth of microorganisms.

3. The method of aspect 1 or 2, wherein obtaining the sample comprises using a microfluidic device to encapsulate the microorganisms in the one or more first droplets of the plurality of droplets.

4. The method of any one of aspects 1-3, wherein the microorganisms grow into a colony inside the one or more first droplets of the plurality of droplets.

5. The method of any one of aspects 1-4, wherein the one or more first droplets of the plurality of droplets are emulsified into an engineered oil.

6. The method of any one of aspects 1-5, wherein the plurality of droplets comprises one or more second droplets of the plurality of droplets that do not contain a microorganism, and wherein the one or more second droplets of the plurality of droplets are emulsified into an engineered oil.

7. The method of aspect 6, wherein the trained artificial intelligence program was trained by a method comprising:
analyzing one or more images, wherein each of the one or more images includes at least one of one or more droplets encapsulating microorganisms of a plurality of droplets and at least one of one or more droplets that do not contain a microorganism of a plurality of droplets, wherein the artificial intelligence is based on machine learning algorithms including neural networks and deep learning, and wherein the artificial intelligence learns whether a droplet is of the one or more droplets encapsulating microorganisms of the plurality of droplets or is of the one or more droplets that do not contain a microorganism of the plurality of droplets.

8. The method of aspect 7, wherein performing the image analysis using the artificial intelligence on the one or more images comprises:
the artificial intelligence determining that the one or more droplets encapsulating microorganisms of the plurality of droplets contain one or more microorganisms and the one or more droplets that do not contain a microorganism of the plurality of droplets do not contain a microorganism.

9. The method of aspect 7 or 8, wherein the artificial intelligence determines the volume of at least one of the one or more droplets encapsulating microorganisms of the plurality of droplets and the volume of at least one of the one or more droplets that do not contain a microorganism of the plurality of droplets.

10. The method of any one of aspects 7-9, wherein the one or more images includes the plurality of droplets, and the artificial intelligence determines a quantity of the one or more droplets encapsulating microorganisms of the plurality of droplets and a quantity of the one or more droplets that do not contain a microorganism of the plurality of droplets from the one or more images of the plurality of droplets.

11. The method of aspect 10, wherein the artificial intelligence learns how many microorganisms are in a droplet containing at least one microorganism, and wherein the artificial intelligence determines a quantity of microorganisms in each of the one or more droplets encapsulating microorganisms of the plurality of droplets.

12. The method of aspect 11, wherein the artificial intelligence learns how many microorganisms initiated a colony, and wherein the artificial intelligence determines a quantity of microorganisms that initiated a colony by comparing one or more images of the plurality of droplets taken at predetermined time intervals.

13. The method of any one of aspects 1-12, wherein quantifying the microorganisms further comprises:
analyzing the one or more first droplets using a digital polymerase chain reaction (dPCR).

14. A method of analyzing food, the method comprising:
obtaining from the food a sample containing microorganisms; and
quantifying microorganisms in the sample according to any one of aspects 1-13.

15. The method of aspect 14, wherein the microorganism is pathogenic.

16. The method of aspect 14, wherein the microorganism is probiotic.

17. A method of diagnosing an infection within a human subject, the method comprising:
obtaining from the human subject a sample containing microorganisms:
quantifying microorganisms in the sample according to any one of aspects 1-13; and
determining that the human subject has an infection when the quantified microorganisms is greater than that of a human subject without an infection.

18. A method of treating a human subject having an infection, the method comprising:
determining that the human subject has an infection according to the method of aspect 17; and
treating the human subject for the infection.

19. A method of analyzing an environmental sample, the method comprising:
obtaining from the environmental sample a sample containing microorganisms: and
quantifying microorganisms in the sample according to any one of aspects 1-13.

20. The method of aspect 19, wherein the environmental sample is water.

21. The method of aspect 19, wherein the environmental sample is sewage.

22. The method of any one of aspects 1-21, wherein the microorganism is bacteria.

It shall be noted that the preceding are merely examples of aspects. Other exemplary aspects are apparent from the entirety of the description herein. It will also be understood by one of ordinary skill in the art that each of these aspects may be used in various combinations with the other aspects provided herein.

The following example should not be construed as in any way limiting the scope of the present disclosure.

### Example 1

This Example demonstrates quantification of droplets with and without bacteria using artificial intelligence, in accordance with aspects of the disclosure.

Droplets of a sample containing bacterial cells were generated using a microfluidic biochip. Once the droplets were generated, a solution with two layers having the emulsion and the oil was collected into an Eppendorf tube at the outlet of the biochip. The Eppendorf tube, which contains droplets with bacterial cells and droplets without bacterial cells, was incubated for 4 hours at 150 rpm to allow bacterial cells to grow inside the droplets with bacterial cells. As a result of the incubation, droplets that are full of bacterial cells and droplets that are empty of bacterial cells can be produced and imaged to distinguish between the full and empty droplets. Then 30 μL of the upper layer, which contains the emulsion with water-in-oil droplets, were taken and injected into a microfluidic flow cell. The flow cell was made of two glass slides, sandwiched with two pieces of double-sided tape placed at the edges to form a capillary channel. Then images of the microfluidic flow cell were obtained by using an optical microscope (e.g., Nikon Eclipse Ti2) coupled with a CCD camera (e.g., Basler acA1920-155 um).

The images were segmented using Image J and the Image Segmenter MATLAB App to analyze the average droplet size and perform the initial classification steps which inputs each image and classifies droplets containing bacteria as opposed to empty droplets. For machine learning analysis, the Statistics and Machine Learning Toolbox from MATLAB was used.

Images were acquired using a 6× phase contrast objective lens. In order to segment the images for droplet size analysis, the Triangle algorithm of Image J was chosen to perform thresholding to divide each image into two or more pixel classes. Based on the camera specifications, the pixel size (H×V) is 5.86 μm, and therefore, the distance in pixels was set at 0.9767 pixels/μm. The average area of the droplets was approximately 3400 μm², and thus, the average diameter of the droplets was calculated to be 65.5 μm.

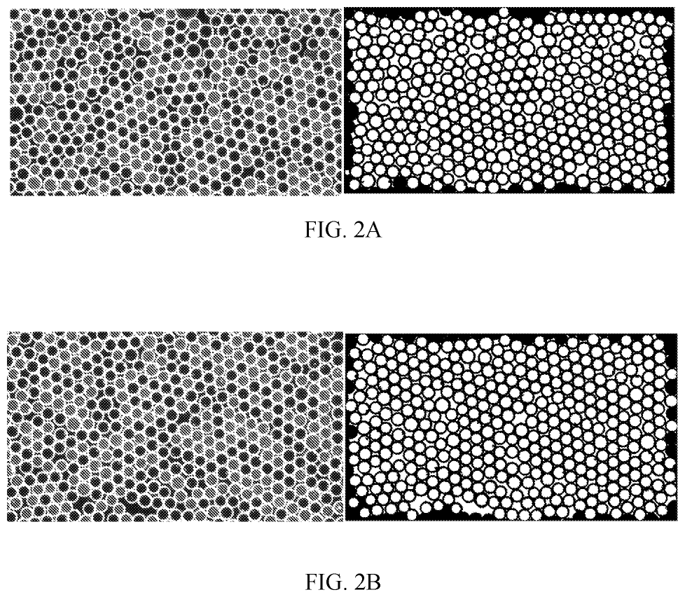

Images were segmented by using an adaptive threshold algorithm of MATLAB App, and closed masks were created with a morphology shaped-disk with a radius of 3. Also, droplets that touched the borders of the images were not included in the analysis. FIGS. 2A and 2B each illustrate on the left: images of droplets full of bacteria (shaded/gray centers) and droplets that are empty (black centers): and on the right: the segmented images of the images on the left. The left and right images of the figures were prepared during separate experiments.

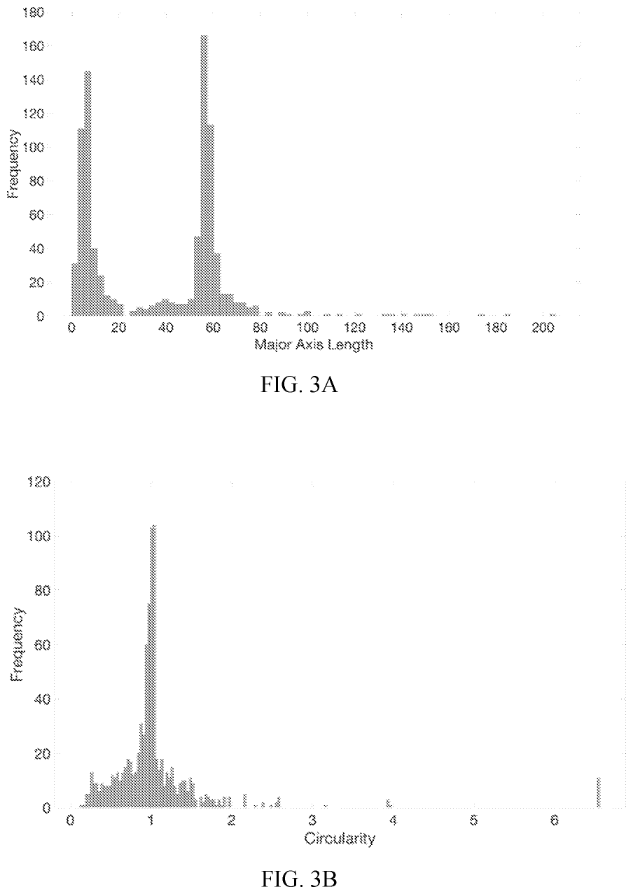

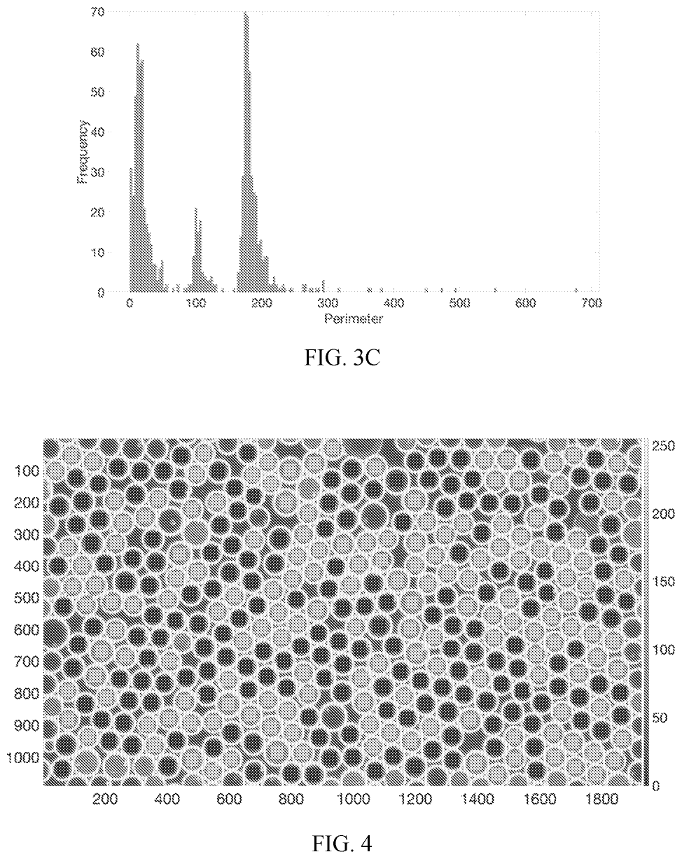

After segmentation, statistical analysis of the droplets was performed based on major axis length, circularity, and perimeter to identify the centroids of the droplets. As illustrated in the histogram of FIG. 3A, the values lower than 20 pixels and greater than 80 pixels of the major axis lengths can be considered noise. However, fewer artifacts with centroids and large droplets were noticed when the range between 50 pixels and 70 pixels was set. FIG. 3B illustrates a histogram of the circularity of the droplets. The circularity value for a perfect circle is 1, and therefore, all values greater than 1 can be removed. Most of the values were between 0.9 and 1.0 and centroids between 0.7 and 1.0 were identified. As illustrated in FIG. 3C, significant portion of the perimeter values ranged between 150 pixels and 250 pixels.

By applying all of these properties as exclusion criteria, greater than 97% of the centroids of the droplets could be identified. Furthermore, a square of 20 pixels (H×V) around each centroid was set in order to determine the mean and median of the intensity of the pixels of each square. As illustrated in FIG. 4, droplets full of bacteria or empty are displayed with differently shaded squares. As a result, the total number of droplets identified by the algorithm was 352, 164 of which corresponded to droplets full of bacteria (47%) and 188 empty droplets (53%).

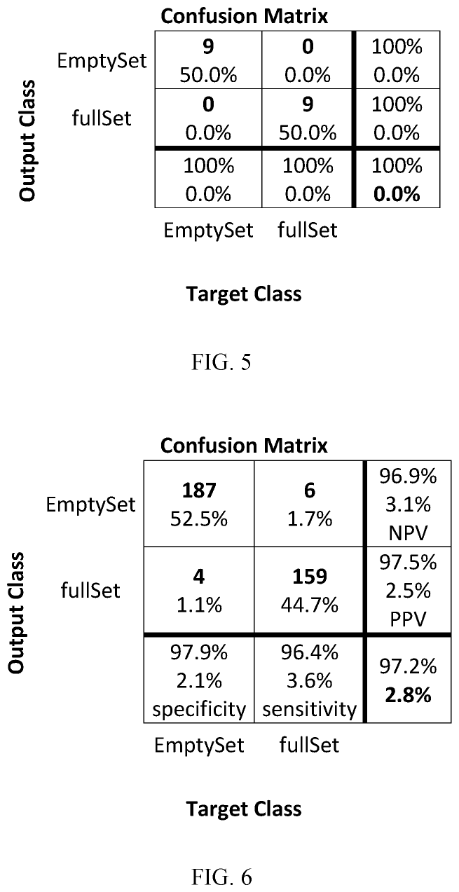

Support Vector Machines were used to classify full droplets and empty droplets. Since there was a low dimensional dataset, fitcsvm was chosen, which is the Train Support Vector Machine classifier used for binary classification. From FIG. 2B, 338 cropped droplet images were obtained and a squared area (20 pixels×20 pixels) around the centroid of each droplet was used to create the model. The size of the training and test dataset was 320 and 18, respectively. FIG. 5 illustrates the confusion matrix for the trained model when the test dataset was used, and all of the 18 test images were classified 100% correctly. After training, the model was used to classify 356 cropped squared images (20 pixels×20 pixels) from FIG. 2A, which contains 191 empty droplets and 165 droplets full of bacteria. As illustrated in FIG. 6, 187 droplets were correctly classified as empty droplets and 6 empty droplets were misclassified as droplets full of bacteria. This corresponds to 52.4% and 1.7%, respectively, of all 356 droplets. Furthermore, the model classified 159 droplets correctly as full of bacteria and misclassified 4 droplets full of bacteria as empty. This corresponds to 44.7% and 1.1%, respectively, of the total number of droplets. Out of the 193 empty droplet prediction, 96.9% were correct and 3.1% were incorrect. Additionally, out of the 163 droplets full of bacteria prediction, 97.5% were correct and 2.5% were incorrect. Hence, out of 191 empty droplets, 97.9% were correctly predicted as empty and 2.1% were predicted as full. Out of 165 droplets full of bacteria, 96.4% were correctly predicted as full and 3.6% were classified as empty. Overall, 97.2% of the predictions were correct and 2.8% were incorrect. As a result, analysis of droplets from FIG. 2A yields an accuracy of 97.2%, a misclassification (error) rate of 2.8%, a sensitivity of 96.4%, a specificity of 97.9%, and a precision of 97.5%.

### Example 2

This Example demonstrates quantification of droplets with and without other types of bacteria using artificial intelligence, in accordance with aspects of the disclosure.

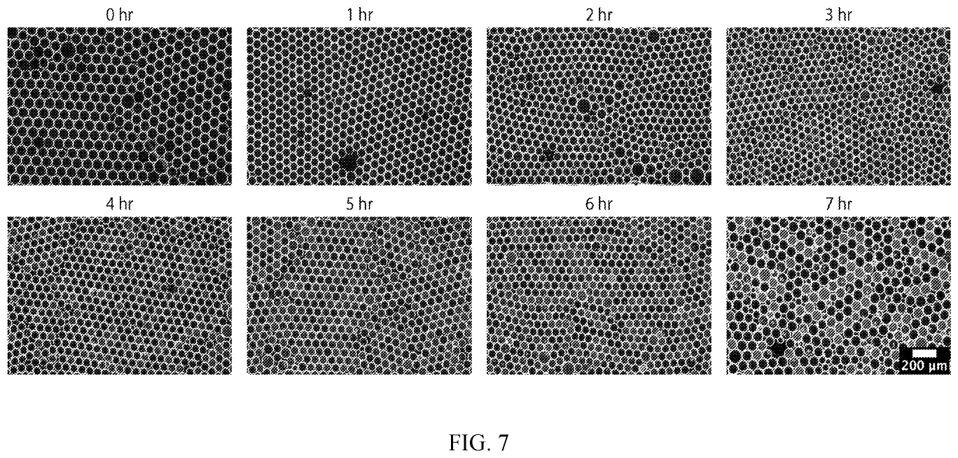

FIG. 7 shows images of droplet cultures to quantify Escherichia coli. A liquid sample containing E. coli at OD=0.4 is emulsified into picoliter-sized droplets using biocompatible fluorinated oils. The initial bacteria concentration is arranged such that roughly 50% of the droplets are occupied with at least one bacterial cell. The droplets are then incubated at 37° C. in an orbital shaker. Droplets are sampled at each hour mark and imaged within a microfluidic flow cell. Droplets with encapsulated bacterial cell(s) ultimately form “droplet colonies”, which are identified by image processing. Due to low magnification imaging, the droplets appear identical at the early stages of incubation. Visual cues of colony formation appear as early as 3 hours where small patches of bacteria become visible. As droplet colonies develop, a notable contrast difference between empty and bacteria containing droplets arise. It was observed that most E. coli droplet colonies become fully developed after 8 hours.

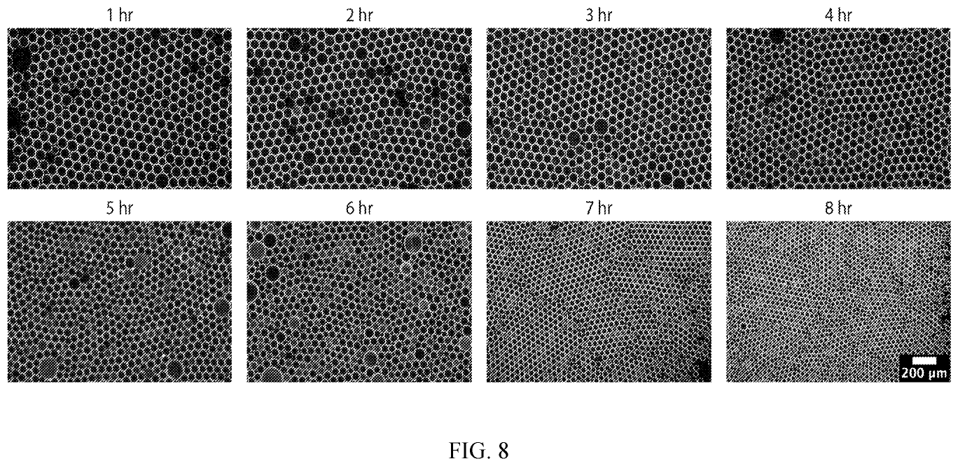

FIG. 8 shows images of droplet cultures to quantify Staphylococcus aureus. A liquid sample containing S. aureus at OD=0.4 is emulsified into picoliter-sized droplets and incubated at 37° C. similar to the E. coli droplet culture. Droplets are sampled at each hour mark and imaged within a microfluidic flow cell. Similar to E. coli droplet culture, obtained were droplet colonies which are visually distinguished from empty droplets using conventional imaging methods. Droplet colonies are fully formed after 7 hours.

These experiments demonstrate that droplet colonies are formed within 8 hours using liquid samples containing E. coli and S. aureus. Formation and detection of droplet colonies within 8 hours presents a significant improvement over existing agar plate based techniques for bacteria enumeration. While droplet colonies form upon sufficient incubation time, all droplets with viable bacterial cells will generate droplet colonies regardless of the initial number of encapsulated bacteria. Thus, end point measurements do not allow differentiating between droplets containing different number of cells at the beginning of the incubation process. This limitation can be addressed by carrying out kinetic measurements, as shown here. Droplet colony images acquired at various time points during the incubation process can be processed using machine learning algorithms to classify droplet occupancy. Maturation of each droplet colony will depend on the initial number of bacteria in each droplet. Therefore, bacterial population within individual droplets at a given time point can be used to generate a more complete representation of droplet statistics. Analysis of the intermediate phases of droplet culture reveals the size of the bacterial colony at a given time, thereby allowing for determination of the initial number of bacteria within individual droplets. To develop a robust quantification scheme, machine learning and artificial intelligence tools are utilized to improve accuracy of this approach for determining initial bacterial concentration.

Demonstration of dCFU by E. coli and S. aureus shows potential applications in clinical microbiology, environmental and food testing.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the present disclosure (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for quantifying microorganisms, the method comprising:
obtaining a sample containing the microorganisms:
encapsulating the microorganisms in one or more first droplets of a plurality of droplets:
allowing microorganism growth within the one or more first droplets of the plurality of droplets:
capturing one or more images of the plurality of droplets:
performing an image analysis using artificial intelligence on the one or more images, wherein the artificial intelligence was trained; and
quantifying the microorganisms.

2. The method of claim 1, wherein the plurality of droplets comprises an aqueous medium containing nutrients nurturing growth of microorganisms.

3. The method of claim 1, wherein obtaining the sample comprises using a microfluidic device to encapsulate the microorganisms in the one or more first droplets of the plurality of droplets.

4. The method of claim 1, wherein the microorganisms grow into a colony inside the one or more first droplets of the plurality of droplets.

5. The method of claim 1, wherein the one or more first droplets of the plurality of droplets are emulsified into an engineered oil.

6. The method of claim 1, wherein the plurality of droplets comprises one or more second droplets of the plurality of droplets that do not contain a microorganism, and wherein the one or more second droplets of the plurality of droplets are emulsified into an engineered oil.

7. The method of claim 6, wherein the trained artificial intelligence program was trained by a method comprising:
analyzing one or more images, wherein each of the one or more images includes at least one of one or more droplets encapsulating microorganisms of a plurality of droplets and at least one of one or more droplets that do not contain a microorganism of a plurality of droplets, wherein the artificial intelligence is based on machine learning algorithms including neural networks and deep learning, and wherein the artificial intelligence learns whether a droplet is of the one or more droplets encapsulating microorganisms of the plurality of droplets or is of the one or more droplets that do not contain a microorganism of the plurality of droplets.

8. The method of claim 7, wherein performing the image analysis using the artificial intelligence on the one or more images comprises:
the artificial intelligence determining that the one or more droplets encapsulating microorganisms of the plurality of droplets contain one or more microorganisms and the one or more droplets that do not contain a microorganism of the plurality of droplets do not contain a microorganism.

9. The method of claim 7, wherein the artificial intelligence determines the volume of at least one of the one or more droplets encapsulating microorganisms of the plurality of droplets and the volume of at least one of the one or more droplets that do not contain a microorganism of the plurality of droplets.

10. The method of claim 7, wherein the one or more images includes the plurality of droplets, and the artificial intelligence determines a quantity of the one or more droplets encapsulating microorganisms of the plurality of droplets and a quantity of the one or more droplets that do not contain a microorganism of the plurality of droplets from the one or more images of the plurality of droplets.

11. The method of claim 10, wherein the artificial intelligence learns how many microorganisms are in a droplet containing at least one microorganism, and wherein the artificial intelligence determines a quantity of microorganisms in each of the one or more droplets encapsulating microorganisms of the plurality of droplets.

12. The method of claim 11, wherein the artificial intelligence learns how many microorganisms initiated a colony, and wherein the artificial intelligence determines a quantity of microorganisms that initiated a colony by comparing one or more images of the plurality of droplets taken at predetermined time intervals.

13. The method of claim 1, wherein quantifying the microorganisms further comprises:
analyzing the one or more first droplets using a digital polymerase chain reaction (dPCR).

14. A method of analyzing food, the method comprising:
obtaining from the food a sample containing microorganisms; and
quantifying microorganisms in the sample according to claim 1.

15. The method of claim 14, wherein the microorganism is pathogenic or probiotic.

16. (canceled)

17. A method of diagnosing an infection within a human subject, the method comprising:
obtaining from the human subject a sample containing microorganisms:
quantifying microorganisms in the sample according to claim 1; and
determining that the human subject has an infection when the quantified microorganisms is greater than that of a human subject without an infection.

18. A method of treating a human subject having an infection, the method comprising:
determining that the human subject has an infection according to the method of claim 17; and
treating the human subject for the infection.

19. A method of analyzing an environmental sample, the method comprising:
obtaining from the environmental sample a sample containing microorganisms; and
quantifying microorganisms in the sample according to claim 1.

20. The method of claim 19, wherein the environmental sample is water or sewage.

21. (canceled)

22. The method of claim 1, wherein the microorganism is bacteria.
